(19) United States
(12) Patent Application Publication
(10) Pub. No.: US 2019/0026655 A1
(43) Pub. Date: Jan. 24, 2019

(54) Machine Learning System for Patient Similarity

(71) Applicant: Petuum Inc., Pittsburgh, PA (US)

(72) Inventors: Pengtao Xie, Pittsburgh, PA (US); Eric Xing, Pittsburgh, PA (US)

(21) Appl. No.: 16/038,895

(22) Filed: Jul. 18, 2018

Related U.S. Application Data
(60) Provisional application No. 62/534,619, filed on Jul. 19, 2017.

(51) Int. Cl. G06N 99/00; G16H 50/70

(52) U.S. Cl. CPC G06N 99/005; G16H 50/20; G16H 50/70

(57) ABSTRACT

Accordingly, patient similarity measurement system is disclosed. In one embodiment, the patient similarity measurement system includes a diversity-promoting distance metric learning (DPDML) model, wherein said PSM system is configured to perform PSM tasks by receiving inputs of the electronic health records (EHRs) of two patients, and generating an output of a score that indicates the similarity of the two patients. One embodiment provides a method for of performing patient similarity measurement via a diversity-promoting distance metric learning model, comprising receiving inputs of the electronic health records (EHRs) of a first patient and a second patient, and generating an output of a score that indicates the similarity of the first and second patient. Other embodiments are disclosed herein.

### PRIORITY CLAIM AND RELATED APPLICATIONS

This non-provisional application claims priority to U.S. Provisional Application Ser. No. 62/534,619, filed on Jul. 19, 2017 entitled “Machine Learning System for Measuring Patient Similarity” and claims priority to U.S. Provisional Application Ser. No. 62/699,385, filed on Jul. 17, 2018 entitled “Diversity-Promoting and Large-Scale Machine Learning for Healthcare”, wherein the entirety of the U.S. priority applications is incorporated herein by reference for all purposes.

### BACKGROUND

### Field of the Invention

The present invention generally relates to machine learning for healthcare, and more particularly, is directed to a method and system of measuring patient similarity via a diversity-promoting distance metric learning model.

### Prior Art

Patient similarity measurement (PSM), which decides whether two patients are similar or dissimilar based on their electronic health records (EHRs), is a critical task for patient cohort identification and finds wide applications in clinical decision-making. For instance, with an effective similarity measure in hand, one can perform case-based retrieval of similar patients for a target patient, who can be subsequently diagnosed and treated by synthesizing the diagnosis outcomes and treatment courses of the retrieved patients. Other applications powered by patient similarity include classification of epidemiological data on hepatic steatosis, patient risk prediction, personalized treatment for hypercholesterolemia, personalized mortality prediction, near-term prognosis, to name a few.

In clinical practice, it is often the case that the frequency of diseases is highly imbalanced, which usually follows a power-law distribution where a small number of diseases have very high frequency while most diseases have low frequency. Due to the skewness of disease frequency, conventional approaches typically are less capable of effectively measuring the similarities between patients who have an infrequent disease. Infrequent diseases are of vital importance and should not be neglected. First, many infrequent diseases are life threatening such as flail chest. Ignoring them would place huge risk over patients. Second, the total amount of infrequent disease is very large. Though the frequency of each infrequent disease is low, the total number of patients diagnosed with infrequent diseases is large because of the large total number of infrequent diseases.

### SUMMARY

Accordingly, patient similarity measurement system is disclosed. In one embodiment, the patient similarity measurement system includes a diversity-promoting distance metric learning (DPDML) model, wherein said PSM system is configured to perform PSM tasks by receiving inputs of the electronic health records (EHRs) of two patients, and generating an output of a score that indicates the similarity of the two patients. One embodiment provides a method for of performing patient similarity measurement via a diversity-promoting distance metric learning model, comprising receiving inputs of the electronic health records (EHRs) of a first patient and a second patient, and generating an output of a score that indicates the similarity of the first and second patient. Other embodiments are disclosed herein.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

The present disclosure, in accordance with one or more various embodiments, is described in detail with reference to the following figures. The drawings are provided for purposes of illustration only and merely depict exemplary embodiments of the disclosure. These drawings are provided to facilitate the reader's understanding of the disclosure and should not be considered limiting of the breadth, scope, or applicability of the disclosure. It should be noted that for clarity and ease of illustration these drawings are not necessarily made to scale.

FIG. 1 provides an overview of a Patient Similarity Measurement (PSM) system comprising four sub-modules according to embodiments of the invention.

FIG. 2 shows the Electronic Health Record (EHR) encoding sub-module of FIG. 1, which comprises four encoding sub-modules that encode four modalities of data and a fusion sub-module that combines the representations of individual modalities into a holistic one according to embodiments of the invention; and

FIG. 3 is a flowchart diagram illustrating an exemplary process underlying the PSM system of FIG. 1 according to embodiments of the invention.

### DETAILED DESCRIPTION

The following description is presented to enable a person of ordinary skill in the art to make and use the invention. Descriptions of specific devices, techniques, and applications are provided only as examples. Various modifications to the examples described herein will be clear to those of ordinary skill in the art, and the general principles defined herein may be applied to other examples and applications without departing from the spirit and scope of the invention. Thus, embodiments of the present invention are not intended to be limited to the examples described herein and shown, but is to be accorded the scope consistent with the claims.

The invention can be implemented in numerous ways, including as a method; a process; an apparatus; a system; a device; a computer hardware or software; or a computing system, method or process implemented through executing computer software instructions; computer software stored in a non-transitory computer-readable storage medium storing instructions, when executed by a computing device or a cluster of computing devices implements the method or process of the invention.

The word “exemplary” is used herein to mean “serving as an example or illustration.” Any aspect or design described herein as “exemplary” is not necessarily to be construed as preferred or advantageous over other aspects or designs.

It is noted that, as used herein, “a” and “an” each generally denotes “at least one,” but does not exclude a plurality unless the contextual use dictates otherwise. Thus, reference to “a car having a car seat” describes “a car having at least one car seat” as well as “a car having car seats.” In contrast, reference to “a car having a single car seat” describes “a car having only one car seat.”

Reference will now be made in detail to aspects of the subject technology, examples of which are illustrated in the accompanying drawings, wherein like reference numerals refer to like elements throughout.

The specific order or hierarchy of steps in the processes disclosed herein is an example of exemplary approaches. Based upon design preferences, it is understood that the specific order or hierarchy of steps in the processes may be rearranged while remaining within the scope of the present disclosure. The accompanying method claims present elements of the various steps in a sample order, and are not meant to be limited to the specific order or hierarchy presented

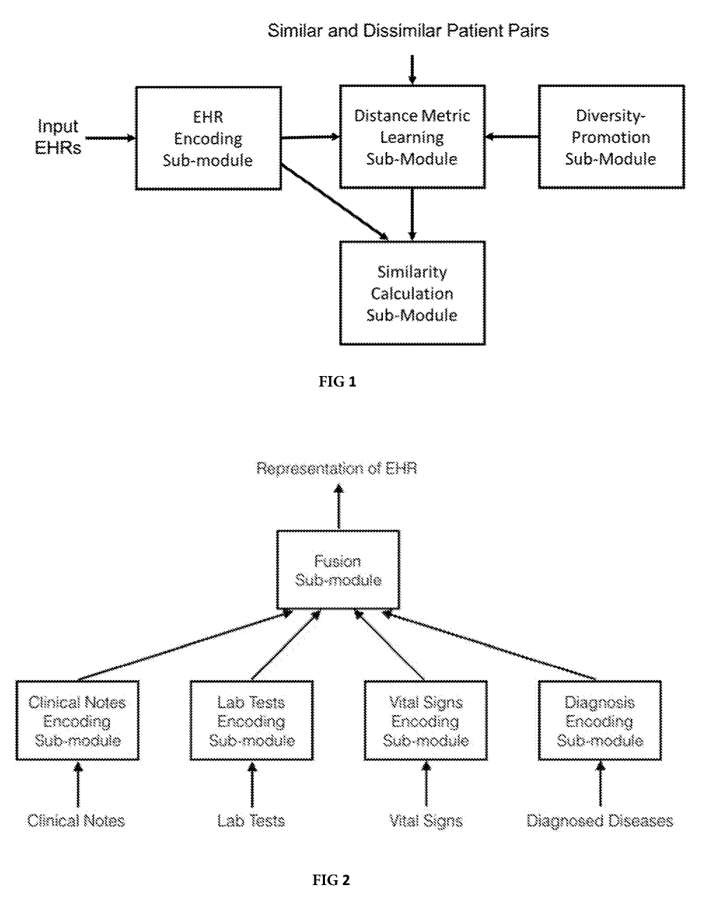

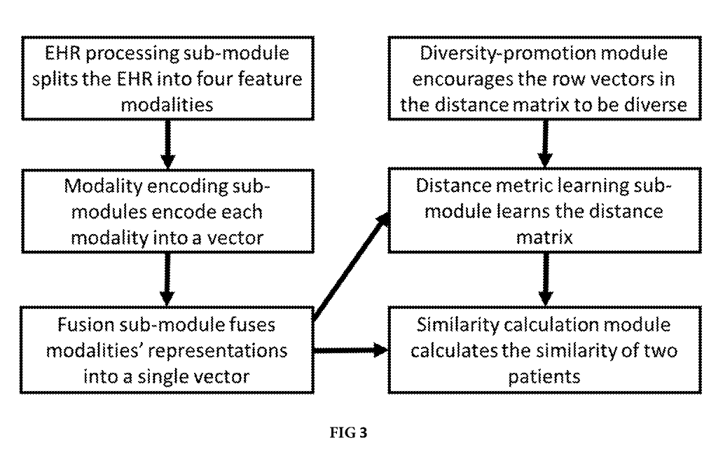

Described herein is a Patient Similarity Measurement (PSM) system, which comprises a Diversity-Promoting Distance Metric Learning (DPDML) model according to embodiments of the invention. Such a PSM system is configured to perform Patient Similarity Measurement tasks, as shown in FIG. 2, by receiving inputs of the electronic health records (EHRs) of two patients and generating an output of a score that indicates the similarity between the two patients. The PSM system learns representations for patients' EHRs, and computes the similarity of the representations in a latent space. Various embodiments of a PSM system use a diversity-promoting distance metric learning model to measure similarity and to capture the similar characteristics of patients having infrequent diseases.

As shown in FIG. 1, a PSM system contains a Diversity-Promoting Distance Metric Learning (DPDML) module. The module takes the EHRs of two patients as inputs, and produces a score that indicates how similar the two patients are. In one embodiment, as shown in FIG. 1, the DPDML module comprises several sub-modules.

In this example, an EHR encoding sub-module learns representation vectors for EHRs. Patient pairs that are labeled by physicians either as similar or dissimilar and the representations of these patients' EHRs are input to the distance metric learning sub-module to learn a distance metric. The distance metric is featured by a projection matrix where the row vectors of this matrix project the representation vectors of patients' EHRs into a lower-dimensional latent space. For example, “diversity” may be characterized by considering two factors: uncorrelation and evenness. In this way, uncorrelation may be a measure of how uncorrelated components are. That is, less correlation is equivalent to more diversity. Additionally, for evenness in latent space modeling, components may play substantially equally important roles and no one component dominating, such that each component contributes significantly in data modeling.

In some embodiments, uncorrelation among components may be characterized from a statistical perspective by treating components as random variables and measuring their covariance which is proportional to their correlation. In one embodiment, A ϵ [symbol] ^{d×m} denotes the component matrix whose k-th column is the parameter vector aₖ of component k. In some embodiments, a row view of A: may be used where each component is treated as a random variable and each row vector ãᵢ^{T} is a sample drawn from the random vector formed by the m components. Further,

$$μ = \frac{1}{d} {Σ}_{i = 1}^{d} {\tilde{a}}_{i} = \frac{1}{d} {A}^{T} 1$$

may be set as the sample mean, where the elements of 1 ϵ [symbol] ^{d} are all 1. An empirical covariance matrix may then be computed with the components as

$$G = \frac{1}{d} {Σ}_{i = 1}^{d} ( {\tilde{a}}_{i} - μ ) {( {\tilde{a}}_{i} - μ )}^{T} = \frac{1}{d} {A}^{T} A - ( \frac{1}{d} {A}^{T} 1 ) {( \frac{1}{d} {A}^{T} 1 )}^{T} .$$

By imposing the constraint A^{T}1=0, therefore

$$G = \frac{1}{d} {A}^{T} A .$$

suppose H is a run rank matrix and m>d, then G is a full-rank matrix with rank m.

For the next step, the eigenvalues of G play important roles in characterizing the uncorrelation and evenness of components. Let G=Σₖ₌₁^{m}λₖuₖuₖ^{T} be the eigendecomposition where λₖ is an eigenvalue and uₖ is the associated eigenvector. In Principle Component Analysis, an eigenvector uₖ of the covariance matrix G represents a principal direction of the data points and the associated eigenvalue λₖ tells the variability of points along that direction. The larger λₖ is, the more spread out the points along the direction uₖ. When the eigenvectors (principal directions) are not aligned with the coordinate axis, the level of disparity among eigenvalues indicates the level of correlation among the m components (random variables). The more different the eigenvalues are, the higher the correlation is. Considering this, the uniformity among eigenvalues of G can be utilized to measure how uncorrelated the components are.

Secondly, the eigenvalues are related with the other factor of diversity: evenness. When the eigenvectors are aligned with the coordinate axis, the components are uncorrelated. In this case, evenness is used to measure diversity. In this example, each component is assigned an importance score. Since the eigenvectors are in parallel to the coordinate axis, the eigenvalues reflect the variance of components. Analogous to PCA which posits that random variables with larger variance are more important, the present embodiment may use variance to measure importance. According to the evenness criteria, the components are more diverse if their importance scores match, which motivates us to encourage the eigenvalues to be uniform.

To sum up, the eigenvalues are encouraged to be even in both cases: (1) when the eigenvectors are not aligned with the coordinate axis, they are preferred to be even to reduce the correlation of components; (2) when the eigenvectors are aligned with the coordinate axis, they are encouraged to be even such that different components contribute equally in modeling data.

Referring to FIG. 1, the similarity (or distance) of patients is then defined in the latent space. Further, row vectors of the projection matrix are encouraged to be diverse by a diversity-promotion sub-module. In some embodiments, by promoting diversity the row vectors evenly spread out and to represent both frequent diseases and infrequent diseases. By doing this, the similarity of patients with infrequent diseases can be better measured as the distance learning model counters skew toward frequent diseases. Given two previously unseen patients, the representations of their EHRs, as produced by an EHR encoding sub-module, and the learned distance metric, are input to the similarity calculation sub-module to calculate the similarity score between the two patients.

The PSM system uses the EHR encoding (EE) sub-module to learn feature representations of input EHRs, which may contain multiple modalities of clinical information, including clinical notes, lab tests, vital signs, demographics, etc. As shown in FIG. 2, one embodiment EE sub-module comprises four encoding sub-modules that encode four modalities of data and a fusion sub-module that combines the representations of individual modalities into a holistic one. Other embodiments may encode other numbers of modalities. In this embodiment EE sub-module, a clinical note encoding sub-module is a convolutional neural network that is designed to capture local correlations among adjacent words and long-range semantics.

Additionally, in this embodiment, the lab tests and vital signs encoding sub-module are long short-term memory networks that are able to capture the temporal structure among lab tests and vital signs. In the present embodiment, the diagnosis-encoding sub-module is a feedforward network that captures non-linear relations among diseases. In other embodiments, sub-modules may use different neural networks, long short-term memory networks and feedforward networks, or even other networks.

In some embodiments, the distance metric learning (DML) sub-module learns a distance metric. It takes patient pairs either labeled as similar or dissimilar by the physicians and the representation vectors of patients' EHRs produced by the EE sub-module as inputs and produces a distance metric that can be utilized to measure the similarity of two patients. The distance metric between two patients is defined in the following way: given the representations of their EHRs, a linear projection matrix is utilized to project these representations into a latent space; then the squared Euclidean distance between the latent representations is measured. The DML sub-module learns this distance metric (specifically, the linear projection matrix) by encouraging the distance between similar patients to be as small as possible, and encouraging the distance between dissimilar patients to be separated by a margin.

The diversity-promotion (DP) sub-module is utilized to control the row vectors of the distance matrix in the DML sub-module, such that these vectors are diverse. In this embodiment, by promoting diversity the row vectors spread out and give frequent diseases and infrequent diseases a fair treatment. In this way, the similarity of patients with infrequent diseases can be better measured. Diversity is measured using near-orthogonality: vectors that are close to being orthogonal are more diverse. To encourage near-orthogonality, the DP sub-module computes the Gram matrix of the row vectors, then encourages the Gram matrix to be close to an identity matrix where the closeness is measured using Bregman matrix divergence.

The similarity calculation (SC) sub-module calculates the similarity of two patients. It takes the representation vectors produced by the EE sub-module as input and produces a score that indicates the similarity of the two patients. At the core of this sub-module is a distance matrix (learned by the DML sub-module) where the row vectors of this matrix project the representation vectors of patients' EHRs into a lower-dimensional latent space. The similarity of patients is then measured in the latent space.

In some embodiments, to promote uniformity among eigenvalues, as a general approach, eigenvalues may be normalized into a probability simplex and then the discrete distribution parameterized by the normalized eigenvalues may be encouraged to have small Kullback-Leibler (KL) divergence with the uniform distribution. Given the eigenvalues {λₖ}ₖ₌₁^{m}, they are then normalized into a probability simplex

$${\hat{λ}}_{k} = \frac{{λ}_{k}}{{Σ}_{j = 1}^{m} {λ}_{k}}$$

based on which a distribution is defined on a discrete random variable X=1, . . . , m where p(X=k)=\hat{λ}ₖ.

In addition, to ensure the eigenvalues are strictly positive, A^{T}A may be set to be positive definite. To encourage {λₖ}ₖ₌₁^{m} to be uniform, the distribution p(X) is set be “close” to a uniform distribution

$$q ( X = k ) = \frac{1}{m} ,$$

where the “closeness” is measured using KL divergence KL(p∥q):

$${Σ}_{k = 1}^{m} {\hat{λ}}_{k} log \frac{{\hat{λ}}_{k}}{1 \text{/} m} = \frac{{Σ}_{k = 1}^{m} {λ}_{k} log {λ}_{k}}{{Σ}_{j = 1}^{m} {λ}_{j}} - log {Σ}_{j = 1}^{m} {λ}_{j} + log m .$$

In this equation, Σₖ₌₁^{m}λₖ log λₖ is equivalent to

$$tr ( ( \frac{1}{d} {A}^{T} A ) log ( \frac{1}{d} {A}^{T} A ) ) ,$$

where log(·) denotes matrix logarithm. To show this, note that

$$log ( \frac{1}{d} {A}^{T} A ) = {Σ}_{k = 1}^{m} log ( {λ}_{k} ) {u}_{k} {u}_{k}^{T} ,$$

according to the property of matrix logarithm. Then,

$$tr ( ( \frac{1}{d} {A}^{T} A ) log ( \frac{1}{d} {A}^{T} A ) )$$

is equal to tr((Σₖ₌₁^{m}λₖuₖuₖ^{T})(Σₖ₌₁^{m} log(λ)k uₖuₖ^{T})) which equals to Σₖ₌₁^{m}λₖ log λₖ. According to the property of trace,

$$tr ( \frac{1}{d} {A}^{T} A ) = {Σ}_{k = 1}^{m} {λ}_{k} .$$

Then the KL divergence can be turned into a diversity-promoting uniform eigenvalue regularizer (UER):

$$\frac{tr ( ( \frac{1}{d} {A}^{T} A ) log ( \frac{1}{d} {A}^{T} A ) )}{tr ( \frac{1}{d} {A}^{T} A )} - log tr ( \frac{1}{d} {A}^{T} A ) ,$$

subject to A^{T}A [symbol] 0 and A^{T}1=0.

UER then may be applied to promote diversity. For example, let [symbol] (A) denote the objective function of an ML model, then a UE-regularized ML problem can be defined as

$${min}_{A} ℒ ( A ) + λ ( \frac{tr ( ( \frac{1}{d} {A}^{T} A ) log ( \frac{1}{d} {A}^{T} A ) )}{tr ( \frac{1}{d} {A}^{T} A )} - log tr ( \frac{1}{d} {A}^{T} A ) )$$

subject to A^{T}A [symbol] 0 and A^{T}1=0, where λ is the regularization parameter.

Uniform eigenvalue regularizers may then be applied to promote diversity in a specific model: distance metric learning (DML). Given data pairs either labeled as “similar” or “dissimilar”, DML aims to learn a distance metric under which similar pairs would be placed close to each other and dissimilar pairs are separated apart. The learned distance can benefit a wide range of tasks, including retrieval, clustering and classification. The distance metric may be defined as between x, y ϵ [symbol] ^{d} as ∥A^{T}x−A^{T}y∥₂² where A ϵ [symbol] ^{d×m} is a parameter matrix whose column vectors are components. A uniform eigenvalue regularized DML (UE-DML) problem can then be formulated as:

$${min}_{A} {Σ}_{( x , y ) {∈}^{𝒮}} \vert \vert {A}^{T} x - {A}^{T} y {\vert \vert}_{2}^{2} + {Σ}_{( x , y ) {∈}^{𝒟}} max ( 0 , 1 - \vert \vert {A}^{T} x - {A}^{T} y {\vert \vert}_{2}^{2} ) + λ ( \frac{tr ( ( \frac{1}{d} {A}^{T} A ) log ( \frac{1}{d} {A}^{T} A ) )}{tr ( \frac{1}{d} {A}^{T} A )} - log tr ( \frac{1}{d} {A}^{T} A ) ) subject to {A}^{T} A ≻ 0 and {A}^{T} 1 = 0 ,$$

where [symbol] and [symbol] are the set of similar and dissimilar pairs respectively. The first and second term in the objective function encourage similar pairs to have small distances and dissimilar pairs to have large distances respectively.

The UE regularizer is nonconvex and is difficult to be convexified. As a result, the UE-regularized ML problems are nonconvex where achieving the global optimal is NP-hard. In this section, diversity-promoting regularizers are designed that make convex relaxation easier. Nonconvex regularizers are defined based on Bregman matrix divergence, then discuss how to convexify them.

With reference to FIG. 1, diversity may also be defined as near-orthogonality, wherein component vectors are determined to be more diverse if they are closer to being orthogonal. To encourage near orthogonality between two vectors aᵢ and aⱼ, one way is to make their inner product aᵢ T aⱼ close to zero and their [symbol] ₂ norm ∥aᵢ∥₂, ∥aⱼ∥₂ close to one. For a set of vectors {aᵢ}ᵢ₌₁^{m}, near orthogonality can be achieved in the following manner by computing the Gram matrix G where Gᵢⱼ=aᵢ^{T}aⱼ, then encouraging G to be close to an identity matrix. Off the diagonal of G and I are aᵢ^{T}aⱼ and zero respectively. On the diagonal of G and I are ∥aᵢ∥₂² and one respectively. Making G close to I effectively encourages aᵢ^{T}aⱼ to be close to zero and ∥aᵢ∥₂ close to one, which therefore encourages aᵢ and aⱼ to get close to orthogonal.

The present embodiment uses Bregman matrix divergence (BMD) to measure “closeness” between two matrices. Let [symbol] ⁿ denote real symmetric n×n matrices. Given a strictly convex, differentiable function ϕ: [symbol] ⁿ→ [symbol] , the BMD is defined as D_{ϕ}(X, Y)=ϕ(X)−ϕ(Y)−tr ((∇ϕ(Y))^{T}(X−Y)), where tr(A) denotes the trace of matrix A. Different choices of ϕ(X) lead to different divergences. When ϕ(X)=∥X∥_{F}², BMD is specialized to the squared Frobenius norm (SFN) ∥X−Y∥_{F}². If ϕ(X)=tr(X log X−X), where log X denotes the matrix logarithm of X, the divergence becomes D_{vN}(X, Y) =tr(X log X−X log Y−X+Y), which is von Neumann divergence (VND). If ϕ(X)=−log detX where det(X) denotes the determinant of X, the log-determinant divergence (LDD) D_{ID}(X, Y)=tr(XY⁻¹) −log det(XY⁻¹)−n.

To encourage near-orthogonality among components, the BMD between the Gram matrix AA^{T} and an identity matrix I may be small, which results in a family of BMD regularizers: ω_{ϕ}(A)=(AA^{T}, I). Ω_{ϕ}(A) can be specialized to different instances, according to the choices of D_{ϕ}(·,·). Under SFN, Ω_{ϕ}(A) becomes Ω_{Fro}(A)=∥AA^{T}−I∥_{F}². Under VND, Ω_{ϕ}(A) becomes Ω_vN(A)=tr(AÂT log(AÂT)−AÂT)+m. Under LDD, Ω_{ϕ}(A) becomes Ω_{ld}(A)=tr(AA^{T})−log det(AA^{T})−m.

Applying these regularizers to distance metric learning (DML), the following BMD-regularized DML (BMD-DML) problem is defined as:

$${min}_{A} \frac{1}{\vert \vert} {Σ}_{( x , y ) ∈} \vert \vert Ax - Ay {\vert \vert}_{2}^{2} + \\ \frac{1}{\vert \vert} {Σ}_{( x , y ) ∈} max ( 0 , 1 - \vert \vert Ax - Ay {\vert \vert}_{2}^{2} ) + {λΩ}_{φ ( A )}$$

which is nonconvex.

While various embodiments of the invention have been described above, they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations.

Additionally, although the disclosure is described above in terms of various exemplary embodiments and implementations, the various features and functionality described in one or more of the individual embodiments are not limited in their applicability to the particular embodiment with which they are described. They instead can be applied alone or in some combination, to one or more of the other embodiments of the disclosure, whether or not such embodiments are described, and if such features are presented as being a part of a described embodiment. Thus, the breadth and scope of the present disclosure should not be limited by any of the above-described exemplary embodiments.

In this document, the terms “module” and “engine” as used herein, refers to software, firmware, hardware, and any combination of these elements for performing the associated functions described herein. Additionally, for purpose of discussion, the various modules are described as discrete modules; however, as would be apparent to one of ordinary skill in the art, two or more modules may be combined to form a single module that performs the associated functions according embodiments of the invention.

In this document, the terms “computer program product”, “computer-readable medium”, and the like, may be used generally to refer to media such as, memory storage devices, or storage unit. These, and other forms of computer-readable media, may be involved in storing one or more instructions for use by processor to cause the processor to perform specified operations. Such instructions, generally referred to as “computer program code” (which may be grouped in the form of computer programs or other groupings), when executed, enable the computing system.

It will be appreciated that, for clarity purposes, the above description has described embodiments of the invention with reference to different functional units and processors. However, it will be apparent that any suitable distribution of functionality between different functional units, processors or domains may be used without detracting from the invention. For example, functionality illustrated to be performed by separate processors or controllers may be performed by the same processor or controller. Hence, references to specific functional units are only to be references to suitable means for providing the described functionality, rather than indicative of a strict logical or physical structure or organization.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; and adjectives such as “conventional,” “traditional,” “normal,” “standard,” “known”, and terms of similar meaning, should not be construed as limiting the item described to a given time period, or to an item available as of a given time. But instead these terms should be read to encompass conventional, traditional, normal, or standard technologies that may be available, known now, or at any time in the future.

Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to”, or other like phrases in some instances shall not be read to mean that the narrower case is intended or required in instances where such broadening phrases may be absent.

Additionally, memory or other storage, as well as communication components, may be employed in embodiments of the invention. It will be appreciated that, for clarity purposes, the above description has described embodiments of the invention with reference to different functional units and processors. However, it will be apparent that any suitable distribution of functionality between different functional units, processing logic elements or domains may be used without detracting from the invention. For example, functionality illustrated to be performed by separate processing logic elements or controllers may be performed by the same processing logic element or controller. Hence, references to specific functional units are only to be seen as references to suitable means for providing the described functionality, rather than indicative of a strict logical or physical structure or organization.

Furthermore, although individually listed, a plurality of means, elements or method steps may be implemented by, for example, a single unit or processing logic element. Additionally, although individual features may be included in different claims, these may possibly be advantageously combined. The inclusion in different claims does not imply that a combination of features is not feasible and/or advantageous. Also, the inclusion of a feature in one category of claims does not imply a limitation to this category, but rather the feature may be equally applicable to other claim categories, as appropriate.

1. A method of performing patient similarity measurement via a diversity-promoting distance metric learning model, said method comprising:
receiving inputs of the electronic health records (EHRs) of a first patient and a second patient; and
generating an output of a score that indicates the similarity of the first and second patient.

2. The method of claim 1, wherein the electronic health records include at least one of clinical notes, lab tests, vital signs, and diagnosed diseases.

3. The method of claim 1, wherein generating an output of a score indicating similarity further includes calculating a distance metric using a projection matrix, where the row vectors of the projection matrix project the representation vectors of patients' EHRs into a lower-dimensional latent space.

4. The method of claim 1, wherein to calculate patient similarity, the method further including calculating uncorrelation between components of inputs of the first patient and inputs of the second patient.

5. The method of claim 4, wherein uncorrelation is calculated using eigenvalues of component matrices composed from the inputs of the first patient EHR and inputs of the second patient EHR, wherein uniformity among the eigenvalues measures uncorrelation between components.

6. The method of claim 6, wherein eigenvalues are promoted to be uniform in order to promote evenness between components.

7. The method of claim 5, further including normalizing the eigenvalues into a probability simplex and encouraging the discrete distribution parameterized by the normalized eigenvalues to have small Kullback-Leibler (KL) divergence with the uniform distribution.

8. The method of claim 7, further comprising calculating a distance metric based on similarity between the normalized eigenvalues between the first patient EHR and the second patient EHR.

9. The method of claim 1, wherein the component vectors are encouraged to be near-orthogonal to promote diversity between the components.

10. A Patient Similarity Measurement (PSM) system comprising:
a Diversity-Promoting Distance Metric Learning (DPDML) model, wherein said PSM system is configured to perform PSM tasks by receiving inputs of the electronic health records (EHRs) of two patients, and generating an output of a score that indicates the similarity of the two patients.

11. The patient similarity measurement system of claim 10, wherein the inputs include at least one of clinical notes, lab tests, vital signs, and diagnosed diseases.

12. The Patient Similarity Measurement (PSM) system of claim 10, further comprising a distance metric learning sub-module to calculate a distance metric using a projection matrix, where the row vectors of the projection matrix project the representation vectors of patients' EHRs into a lower-dimensional latent space.

13. The Patient Similarity Measurement (PSM) system of claim 10, wherein to calculate patient similarity, the system further includes a similarity calculation submodule to calculate uncorrelation between components of inputs of the first patient and inputs of the second patient.

14. The Patient Similarity Measurement (PSM) system of claim 13, wherein uncorrelation is calculated using eigenvalues of component matrices composed from the inputs of the first patient EHR and inputs of the second patient EHR, wherein uniformity among the eigenvalues measures uncorrelation between components.

15. The Patient Similarity Measurement (PSM) system of claim 14, wherein eigenvalues are promoted to be uniform in order to promote evenness between components.

16. The Patient Similarity Measurement (PSM) system of claim 14, further including normalizing the eigenvalues into a probability simplex and encouraging the discrete distribution parameterized by the normalized eigenvalues to have small Kullback-Leibler (KL) divergence with the uniform distribution.

17. The Patient Similarity Measurement (PSM) system of claim 16, further comprising the distance metric learning submodule being configured to calculate a distance metric based on similarity between the normalized eigenvalues between the first patient EHR and the second patient EHR.

18. The Patient Similarity Measurement (PSM) system of claim 10, wherein the component vectors are encouraged to be near-orthogonal to promote diversity between the components.
